(19) United States
(12) Patent Application Publication
(10) Pub. No.: US 2006/0282114 A1
(43) Pub. Date: Dec. 14, 2006

(54) Embolic protection apparatus with vasodilator coating

(75) Inventor: David D. Barone, Lexington, MA (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 11/148,743

(22) Filed: Jun. 9, 2005

(51) Int. Cl. A61M 29/00

(52) U.S. Cl. 606/200

(57) ABSTRACT

An apparatus for temporary prevention of embolization in a human blood vessel. The apparatus comprises a body that is transformable between a radially collapsed configuration and a radially expanded configuration sized and shaped for sealing against an inner wall of the vessel to at least partially obstruct fluid flowing there through, the body having a vasodilator coating.

### FIELD OF THE INVENTION

The present invention relates generally to an intraluminal apparatus for prevention of embolization in a blood vessel during an interventional vascular procedure. More specifically, the invention relates to a catheter having a temporary filter or a temporary occluder.

### BACKGROUND OF THE INVENTION

A variety of therapeutic modalities exists for treating vessel narrowings, which may comprise atherosclerotic plaque and/or thrombus, and which may be alternatively described as stenoses or lesions. Balloon angioplasty, stent deployment, atherectomy, and thrombectomy are well known techniques that have proven effective, alone or in combination, in the treatment of such stenoses. During each of these procedures, there is a risk that particulates dislodged by the procedure will migrate through the circulatory system to embolize in critical organs, causing ischaemic events such as infarction or stroke. Thus, practitioners have approached prevention of escaped emboli through use of occlusion devices, filters, lysing, and aspiration techniques. For example, it is known to remove embolic material by suction through an aspiration lumen in the treatment catheter or by capturing emboli in a filter or with an occlusion device.

Prior art intraluminal filters and occluders for protection against atheroembolization are associated with either a catheter or a guidewire and are temporarily positioned downstream of the area to be treated. The terms “distal” and “proximal” are used herein with respect to the treating clinician. That is, distal or distally refer to being distant from or a direction away from the clinician and proximal or proximally refer to being close to or a direction towards the clinician. A known distal protection device (DPD) includes an inflatable occlusion balloon mounted about the distal end of an elongate catheter, or hollow guidewire shaft. The shaft provides a lumen for inflating and deflating the balloon with a fluid inflation media such as carbon dioxide gas or dilute radiopaque contrast liquid. After temporarily occluding the vessel and blocking the passage of potentially embolic debris, an aspiration catheter may be used to extract contaminated blood proximal to the balloon. The occlusion balloon may then be deflated, permitting normal blood flow to resume. Occlusion devices may be positioned either distally or proximally of the area to be treated.

One prior art filter catheter includes a collapsible filter mounted distally of a dilatation balloon. Filter material is secured to resilient ribs, and a filter balloon is located between the catheter exterior and the ribs. Inflation of the filter balloon extends the ribs outward across the vessel to form a trap for fragments loosened by the dilatation balloon. When the filter balloon is deflated, the resilient ribs retract against the catheter to retain the fragments during withdrawal of the catheter. Another prior art filter includes a filter mounted on the distal portion of a hollow guidewire or tube. A movable core wire is used to open and close the filter. The filter distal end is secured to the guide wire and the proximal end has an expandable rim formed by the core wire.

Other known collapsible intraluminal filters are formed from braided filaments. In some designs, the braided pattern has small interstices, making it unnecessary to cover the braid with a porous membrane or filter material. One example has a cylindrical central body for deployment against the wall of a vessel lumen. Both ends of the braided filter are gathered or tapered for attachment to the shaft of a catheter or guidewire. Holes substantially larger than the interstices are cut in a tapered end of the braided filter to form inlet ports. Alternatively, enlarged inlet ports may be formed by stretching selected interstices around shaping mandrels and heat-treating the filter to set the desired sizes and shapes of the ports.

Collapsible protection devices may comprise a membrane disposed over a supporting structure of flexible struts or braided filaments. In such filters, the membrane is porous, allowing blood, but not particulate debris, to flow through the pores. Filters may be considered to partially obstruct blood flow during use. In occluders, the membrane is non-porous, obstructing blood flow completely. Either type of protection device may be self-expanding into a sealing apposition with the inner surface of the vessel wall. Such self-expanding devices may require an outer sheath to slide over and retain the expandable element in a collapsed configuration during delivery and withdrawal.

A problem associated with the use of embolic protection devices is the inducement of vessel spasm when the device contacts the inner surface of the vessel wall. Vasospasm may include sudden occlusive vasoconstriction of a segment of an artery, resulting in a dramatic reduction of blood flow and complicating the treatment procedure. Vasospasm may be caused by contact between the vessel wall and a foreign body such as the embolic protection device. Spasm may be stimulated by relative movement between the protection device and the vessel as a result of external manipulation of the apparatus. In the case of coronary arteries, such relative movement may result from normal beating of the heart. Therefore, there is a need for an apparatus for temporary prevention of embolization in a blood vessel, wherein the apparatus is adapted to reduce or prevent spasm of the vessel wall during use.

### SUMMARY OF THE INVENTION

An aspect of the present invention comprises an apparatus for temporary prevention of embolization in a human blood vessel. The apparatus comprises a body that is transformable between a radially collapsed configuration and a radially expanded configuration sized and shaped for sealing against an inner wall of the vessel to at least partially obstruct fluid flowing there through, the body having a vasodilator coating to act on the tissues of the vessel wall to reduce or prevent the incidence of spasm.

### BRIEF DESCRIPTION OF THE DRAWINGS

The appended drawings are illustrative of the particular embodiments of the invention and therefore do not limit its scope. They are presented to assist in providing a proper understanding of the invention. The present invention will hereinafter be described in conjunction with the drawings, wherein like reference numerals denote like elements. The drawings are not to scale

FIG. 1 illustrates an apparatus in accordance with the invention, deployed within a portion of the coronary arterial anatomy;

FIG. 2 illustrates a side view of a filter catheter in accordance with the invention, including a slidable sheath;

FIG. 3 illustrates intersecting filaments of a braid in accordance with the invention;

FIG. 4 illustrates a cross-section of a portion of vasodilator-coated membrane supported by a strut in accordance with the invention; and

FIG. 5 illustrates a side view of an occluder guidewire in accordance with the invention.

### DETAILED DESCRIPTION OF THE INVENTION

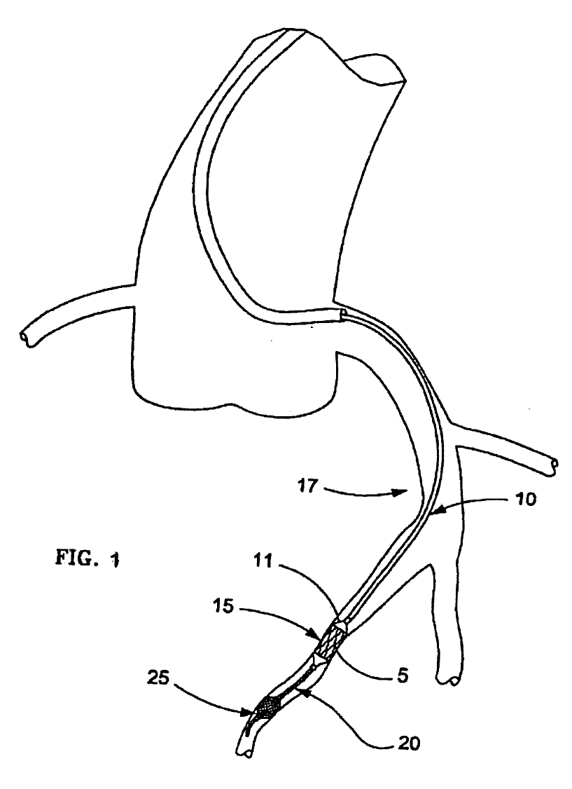

The present invention is a temporary device for use during minimally invasive procedures such as interventional vascular catheterization or other procedures where the practitioner desires to capture embolic material that may be dislodged during the procedure. Intravascular procedures such as PTCA or stent deployment are often preferable to more invasive surgical techniques in the treatment of vascular narrowings, called stenoses or lesions. With reference to FIG. 1, deployment of balloon expandable stent 5 is accomplished by threading catheter 10 through the vascular system of the patient until stent 5 is located within a stenosis at predetermined treatment site 15. Once positioned, balloon 11 of catheter 10 is inflated to expand stent 5 against the vascular wall to maintain the opening. Stent deployment can be performed following treatments such as angioplasty, or during initial balloon dilation of the treatment site, which is referred to as primary stenting.

Catheter 10 is typically guided to treatment site 15 by a guidewire. In cases where the target stenosis is located in tortuous vessels that are remote from the vascular access point, such as coronary arteries 17 shown in FIG. 1, a steerable guidewire is commonly used. In FIG. 1, filter 25 is mounted on such a steerable guidewire. Although FIG. 1 shows inventive protection apparatus 20 and expandable element 25 disposed within coronary artery 17, it should be understood that the invention is applicable to treatments in other vessels of the human body, such as renal, carotid, or other arteries.

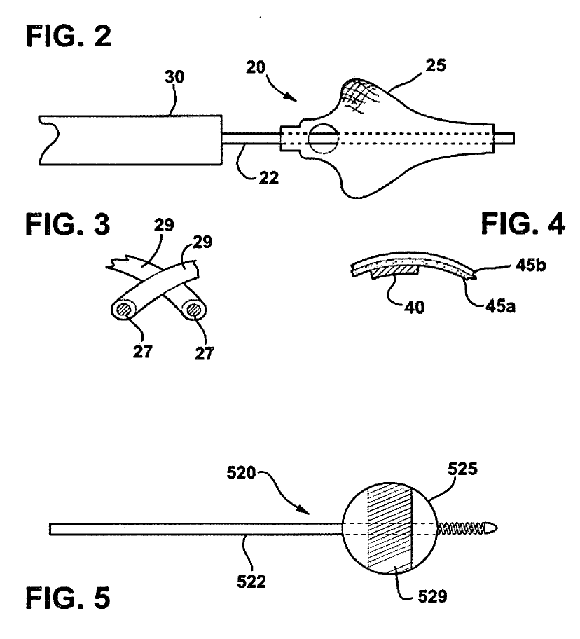

In FIG. 2, protection apparatus 20 is a filter catheter, rather than a filter guidewire. Filter 25 is mounted about the distal end of shaft 22 and is shown in a radially expanded configuration. Filter 25 is self-expanding, meaning that it returns resiliently to the unstressed, expanded configuration. As is well known in the art, sheath 30 may slide over catheter shaft 22 to transform and/or retain filter 25 in a low profile, collapsed configuration (not shown) during delivery and retrieval.

Filter 25 comprises a generally tubular form made from a multiplicity of braided filaments 27. Filter body 25 has a generally constant-pitch braid that is sufficiently dense so that no additional filter material or membrane is necessary for trapping potentially embolic particulates. In one example, 48 filaments can be densely braided to form interstices having a uniform pore size of approximately 75-125 microns. Filaments 27 may be made of a high-modulus polymer or of a metal such as TiNi (nitinol), 316L stainless steel, or cobalt nickel chromium molybdenum superalloy. Additionally, a braiding wire having enhanced radiopacity may be made of, coated with, or coaxially drawn with a radiopaque metal such as gold, platinum, tungsten, alloys thereof, or other biocompatible metals that, compared with stainless steel or nitinol, have a relatively high X-ray attenuation coefficient. One or more filaments having enhanced radiopacity may be inter-woven with non-radiopaque wires, or all wires comprising a protection element may have the same enhanced radiopacity. Filter 25 may be heat set to retain shape memory of the expanded configuration.

Braided filaments 27 are coated with vasodilator 29, as shown in FIG. 3. A vasodilator is a substance that causes blood vessels in the human body to become wider by relaxing the smooth muscle in the vessel wall, or vasodilation. Vasodilator 29 may be selected from a group consisting of adenosine, adrenaline, alpha-blockers, anti-adrenergic drugs, atrial natriuretic peptide, beta-blockers, bradykinin, calcium-antagonists, clonidine, dihydropiridine drugs, fasudil, glycerol trinitrate, guanetidine, histamine, niacin, nicorandil, nifedipine, nitrates, nitric oxide, nitroglycerin, nitroprusside, nesiritide, non dihydropiridine drugs, noradrenaline, papaverine, prostacyclin, prostaglandins, rhokinase inhibitors, tetrahydrocannabinol, and combinations of the above.

Vasodilator 29 may be coated directly onto filaments 27. Alternatively, vasodilator 29 may comprise a pharmaceutical agent blended with a carrier material or applied over a base coat layer to enhance adhesion to filaments 27 or to control the rate of dispersion or elution of the pharmaceutical agent into the tissue of the vessel wall. Such blending or layering techniques are well known in the field of drug-coated stents. The localized delivery of vasodilator to the vessel wall may permit the use of pharmaceutical agents that are too powerful to be safely injected systemically in quantities or concentrations that would be effective in reversing arterial spasm. It may be useful to apply especially powerful vasodilators to only the localized surface of filter 25 that is expected to contact the tissue of the vessel wall. When the apparatus comes into sealing contact with the vessel wall, the mechanism of action is for the vasodilator to be absorbed through the intimal lining of the blood vessel and to act directly on the medial, smooth muscle layer of the vessel, thus reducing or preventing vasospasm.

Alternatively, a membrane may be mounted over braided filaments 27, which are used in this case only as a support structure. In this example, the braid density can be made less dense than is required to form a braided filter. The membrane may be porous or non-porous, depending on whether a filter or an occluder is desired. The membrane may be an elastic or flexible polymer. The membrane support structure may, alternatively, comprise one or more flexible struts 40, as shown in FIG. 4. Struts 40 may have one of a variety of expandable membrane-supporting arrangements, as are well known in the art of distal protection filters. The non-porous membrane shown in FIG. 4 comprises a base layer 45a and a pharmaceutical agent 45b to form a vasodilator-coated occluder.

FIG. 5 illustrates occluder guidewire 520, comprising an elongate hollow shaft 522 and a distally mounted, inflatable occluder balloon 525. Shaft 522 may comprise a guidewire-sized hypotube with a lumen there through for inflating and deflating the balloon with a fluid inflation media such as carbon dioxide gas or dilute radiopaque contrast liquid. As discussed above with regard to filter 25, it may be useful to apply vasodilator 529 to only the selected surface of balloon 525 that is expected to contact the tissue of the vessel wall.

While at least one exemplary embodiment has been presented in the foregoing detailed description, it should be appreciated that numerous variations exist. For example, instead of being normally expanded, the protection apparatus may be normally collapsed, in which case a delivery sheath would not be required. Rather, a mechanical system can be used to expand and collapse the filter or occluder element, as will be understood by those of skill in the art. For purposes of the invention, the filter or occluder element may be mounted on either a catheter shaft or a guidewire. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing the exemplary embodiment or exemplary embodiments. It should be understood that various changes can be made in the function and arrangement of elements without departing from the scope of the invention as set forth in the appended claims and the legal equivalents thereof.

What is claimed is:

1. An apparatus for temporary prevention of embolization in a human blood vessel, the apparatus comprising a body transformable between a radially collapsed configuration and a radially expanded configuration sized and shaped for sealing against an inner wall of the vessel to at least partially obstruct fluid flowing there through, the body having a vasodilator coating.

2. The apparatus of claim 1 wherein the vasodilator is selected from a group consisting of adenosine, adrenaline, alpha-blockers, anti-adrenergic drugs, atrial natriuretic peptide, beta-blockers, bradykinin, calcium-antagonists, clonidine, dihydropiridine drugs, fasudil, glycerol trinitrate, guanetidine, histamine, niacin, nicorandil, nifedipine, nitrates, nitric oxide, nitroglycerin, nitroprusside, nesiritide, non dihydropiridine drugs, noradrenaline, papaverine, prostacyclin, prostaglandins, rhokinase inhibitors, tetrahydrocannabinol, and combinations of the above.

3. The apparatus of claim 1 wherein the vasodilator coating is disposed on at least a surface of the body that is sized and shaped for sealing contact with the inner wall of the vessel.

4. The apparatus of claim 1 wherein the vasodilator coating comprises a pharmaceutical agent and a carrier material.

5. The apparatus of claim 4 wherein the carrier material is a base coat.

6. The apparatus of claim 1 wherein the body is a filter.

7. The apparatus of claim 6 wherein the filter comprises braided filaments.

8. The apparatus of claim 7 wherein the filter comprises a porous membrane disposed on the braided filaments.

9. The apparatus of claim 6 wherein the filter comprises a porous membrane supported by at least one strut.

10. The apparatus of claim 1 wherein the body is an occluder.

11. The apparatus of claim 10 wherein the occluder is an inflatable balloon.

12. The apparatus of claim 10 wherein the occluder comprises a non-porous membrane supported by at least one strut.

13. The apparatus of claim 1 wherein the body is un-stressed in the expanded configuration.

14. The apparatus of claim 1 further comprising an elongate shaft having a distal portion mounted within the body.

15. The apparatus of claim 14 further comprising an elongate sheath slidably disposed about the shaft and being sized to fit over at least a portion of the body when the body is in the collapsed configuration.

16. The apparatus of claim 14 wherein the shaft is a guidewire.
